(19) United States
(12) Patent Application Publication
(10) Pub. No.: US 2020/0345879 A1
(43) Pub. Date: Nov. 5, 2020

(54) GRIP ASSISTING AND ANTI CONTAGION DEVICES

(71) Applicants: Steve RAPP, Rochester, NY (US); Steve LaBUZETTA, Rochester, NY (US)

(72) Inventors: Steve RAPP, Rochester, NY (US); Steve LaBUZETTA, Rochester, NY (US)

(21) Appl. No.: 16/862,955

(22) Filed: Apr. 30, 2020

Related U.S. Application Data
(60) Provisional application No. 62/841,121, filed on Apr. 30, 2019.

(51) Int. Cl. A61L 2/238; A61L 2/26

(52) U.S. Cl. CPC A61L 2/238; A45B 9/02; A61L 2/26

(57) ABSTRACT

A grip assist device fittable to a hand-held object. The grip assist device is comprised of a hollow body having at least one opening for receiving the object. The hollow body is formed of an elastomeric material, and may include an antipathogenic material. The hollow body is comprised of an elastic mesh side wall including perforations interspersed with web sections. The antipathogenic material may be provided in a coating adhered to an external surface of the elastic mesh side wall of the elongated hollow body, and/or impregnated in the elastic mesh side wall of the elongated hollow body. The grip assist device may be provided in a configuration fittable to a walking cane, a door knob, a beverage cup, a handgun, or a bicycle.

### CROSS-REFERENCE TO RELATED PATENT APPLICATIONS

This application claims the benefit of U.S. Provisional Patent Application No. 62/841,121 filed Apr. 30, 2019, the disclosure of which is incorporated herein by reference. The above benefit claim is being made in an Application Data Sheet submitted herewith in accordance with 37 C.F.R. 1.76 (b)(5) and 37 C.F.R. 1.78.

### BACKGROUND

### Technical Field

Ergonomically beneficial gripping devices for retrofitting onto hand-held and hand-operated articles and apparatus, which may also serve to prevent the transmission of pathogens.

### Description of Related Art

There are many hand-held and hand-operated articles and devices that are used by people in daily life activities. Such devices include canes, doorknobs, kitchen utensils, beverage glasses and cups, sports clubs and rackets, walkers, and bicycles. The safe and effective use and operation of these devices depends upon the user having a firm and stable grip upon them. This need is particularly felt by the disabled, elderly and children, who have relatively small hands and/or compromised gripping strength and ability.

These articles and devices are frequently provided by their manufacturers with gripping surfaces that are hard, and with a relatively low coefficient of friction. They do not adjust their respective shapes to conform to the particular user's hand shape, hand size, and gripping technique, and they are not engaged as firmly as desired due to a lack of friction with the user's hand.

The need for a firm and stable grip on a hand-held device, and the action by a user to firmly grip a device in order to operate the device is disadvantageous from the standpoint of hygiene. Many of the above cited exemplary hand-held and hand-operated articles and devices are used by multiple individuals, and in many cases, by the general public. These articles and devices can easily become contaminated with bacteria and viruses such as common cold, flu, and Covid viruses, and serve as a medium for harboring and transmission of infection from one individual to many individuals. In use of these devices, the more firmly they are gripped with greater force and greater skin contact surface area, the more effective the transmission of infection will be.

There is therefore a need for an ergonomically beneficial gripping device that can easily be retrofitted onto a hand-held or hand-operated article or device to provide improved gripping and operation thereof. There is also a need for a gripping device that can easily be retrofitted onto a hand-held or hand-operated article or device, which is provided with antimicrobial and/or antiviral properties, such that it is self-cleaning and disinfecting.

### SUMMARY

In one aspect of the present disclosure, the above needs are met by a grip assist device fittable to a hand-held object. The grip assist device is comprised of a hollow body having at least one opening for receiving the object. The hollow body is formed of an elastomeric material that may include an antipathogenic material. The hollow body is comprised of an elastic mesh side wall including perforations interspersed with web sections. In certain embodiments, the antipathogenic material may be provided in a coating adhered to an external surface of the elastic mesh side wall of the elongated hollow body, and/or impregnated in the elastic mesh side wall of the elongated hollow body. The antipathogenic material may include copper, copper salts, copper alloys and silver.

In another aspect of the present disclosure, a grip assist device fittable to a walking cane is provided. The walking cane is comprised of a handle and a support shaft joined to and extending transversely from the handle. The grip assist device is comprised of an elongated hollow body formed of an elastomeric material and comprised of an elastic mesh side wall including perforations interspersed with web sections, and an opening in the sidewall elastically deformable to receive the handle of the cane within the elongated hollow body and to reversibly deform and surround the transversely extending support shaft of the cane. The elastomeric material of the device may include an antipathogenic material as described above.

In certain embodiments, the grip assist device may include a magnet joined to the elongated hollow body, and/or a luminescent member joined to the elongated hollow body. In certain embodiments, the elastomeric material of the elongated hollow body may be made of a material having a high coefficient of friction. In such embodiments, the elongated hollow body may be further comprised of a lateral protrusion extending outwardly from a side region of the elongated hollow body. In certain embodiments, the elongated body may further comprise a hook feature to facilitate hanging of the cane in a storage position or location when the cane is not in use.

### BRIEF DESCRIPTION OF THE DRAWINGS

The present disclosure will be provided with reference to the following drawings, in which like numerals refer to like elements, and in which:

FIG. 1A is a left side view of a grip assist device fittable to a walking cane;

FIG. 1B is a right side view of the grip assist device of FIG. 1A;

FIG. 1C is a top view of the grip assist device of FIG. 1A;

FIG. 1D is a bottom view of the grip assist device of FIG. 1A;

FIG. 1E is a front end view of the grip assist device of FIG. 1A;

FIG. 1F is a rear end view of the grip assist device of FIG. 1A;

FIG. 1G is a perspective view of the grip assist device of FIG. 1A;

FIG. 1H is the perspective view of FIG. 1G, but with the grip assist device fitted to a walking cane;

FIG. 1I is a right side view of the grip assist device of FIG. 1A with the grip assist device fitted to a walking cane;

FIG. 1J is a the right side view of FIG. 1I, further showing the cane in its entirety;

FIG. 1K is a right side cross-sectional view of the grip assist device of FIG. 1A fitted to a walking cane;

FIG. 2A is a front view of a grip assist device fittable to a door knob;

FIG. 2B is a front right side view of the grip assist device of FIG. 2A, shown rotated about 45 degrees from the front position shown in FIG. 2A;

FIG. 2C is a right side view of the grip assist device of FIG. 2A, shown rotated about an additional 45 degrees from the position shown in FIG. 2B;

FIG. 2D is a top of forward view of the grip assist device of FIG. 2A;

FIG. 2E is a bottom or rearward view of the grip assist device of FIG. 2A;

FIG. 2F is a perspective view of the grip assist device of FIG. 2A;

FIG. 2G is the perspective view of FIG. 2F, but with the grip assist device fitted to a doorknob;

FIG. 3A is a front view of a grip assist device fittable to a beverage cup;

FIG. 3B is a front right side view of the grip assist device of FIG. 3A, shown rotated about 45 degrees from the front position shown in FIG. 3A;

FIG. 3C is a right side view of the grip assist device of FIG. 3A, shown rotated about an additional 45 degrees from the position shown in FIG. 3B;

FIG. 3D is a top view of the grip assist device of FIG. 3A;

FIG. 3E is a bottom view of the grip assist device of FIG. 3A;

FIG. 3F is a perspective view of the grip assist device of FIG. 3A;

FIG. 3G is the perspective view of FIG. 3F, but with the grip assist device fitted to a beverage cup;

FIG. 4A is a left side view of a grip assist device fittable to a handgun;

FIG. 4B is a front view of the grip assist device of FIG. 4A;

FIG. 4C is a right side view of the grip assist device of FIG. 4A;

FIG. 4D is a rear view of the grip assist device of FIG. 4A;

FIG. 4E is a top view of the grip assist device of FIG. 4A;

FIG. 4F is a bottom view of the grip assist device of FIG. 4A;

FIG. 4G is a left side view of the device of FIG. 4A, but with the grip assist device fitted to a handgun;

FIG. 4H is a perspective view of the grip assist device of FIG. 4A;

FIG. 4I is the perspective view of FIG. 4H, but with the grip assist device fitted to a handgun;

FIG. 5A is a front side view of a grip assist device fittable to a bicycle handlebar;

FIG. 5B is a top view of the grip assist device of FIG. 5A;

FIG. 5C is a rear side view of the grip assist device of FIG. 5A;

FIG. 5D is a bottom view of the grip assist device of FIG. 5A;

FIG. 5E is a view of a closed end of the grip assist device of FIG. 5A;

FIG. 5F is a view of an open end of the grip assist device of FIG. 5A;

FIG. 5G is a perspective view of the grip assist device of FIG. 5A;

5H is the perspective view of FIG. 5G, but with the grip assist device fitted to a bicycle handlebar; and

FIG. 5I is a perspective view of a bicycle handlebar showing grip assist devices of FIG. 5A fitted to each end of a bicycle handlebar;

The present invention will be described in connection with certain preferred embodiments. However, it is to be understood that there is no intent to limit the invention to the embodiments described. On the contrary, the intent is to cover all alternatives, modifications, and equivalents as may be included within the spirit and scope of the invention as defined by the appended claims.

### DETAILED DESCRIPTION

For a general understanding of the present invention, reference is made to the drawings. In the drawings, like reference numerals have been used throughout to designate identical elements. The drawings are to be considered exemplary, and are for purposes of illustration only. The dimensions, positions, order and relative sizes reflected in the drawings attached hereto may vary.

In the following disclosure, certain gripping devices may be described using adjectives such as “top,” “upper,” “bottom,” “lower,” “left,” “right,” etc. These adjectives are provided in the context of illustrated uses of the devices and in the context of the orientation of the drawings, which is arbitrary. The description is not to be construed as limiting the gripping devices to use in a particular spatial orientation. The present gripping devices may be used in orientations other than those shown and described herein.

It is also to be understood that any connection references used herein (e.g., attached, coupled, connected, and joined) are to be construed broadly and may include intermediate members between a collection of elements and relative movement between elements unless otherwise indicated. As such, connection references do not necessarily imply that two elements are directly connected and in fixed relation to each other.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the present disclosure.

The term “providing”, such as “providing a gripping device” and the like, when recited in the claims, is not intended to require any particular delivery or receipt of the provided item. Rather, the term “providing” is merely used to recite items that will be referred to in subsequent elements of the claim(s), for purposes of clarity and ease of readability.

The terms “about” and “substantially” are used herein with respect to measurable values and ranges due to expected variations known to those skilled in the art (e.g., limitations and variabilities in measurements).

As used herein, the term “antipathogenic” with reference to a material or object including the material is meant to indicate the presence of a material property that acts against, and may be lethal to pathogens, such as bacteria, viruses, protozoa or fungi. An antipathogenic material as referred to herein is not necessarily lethal to all groups of pathogens, or all individual pathogens among the groups of bacteria, viruses, protozoa and fungi.

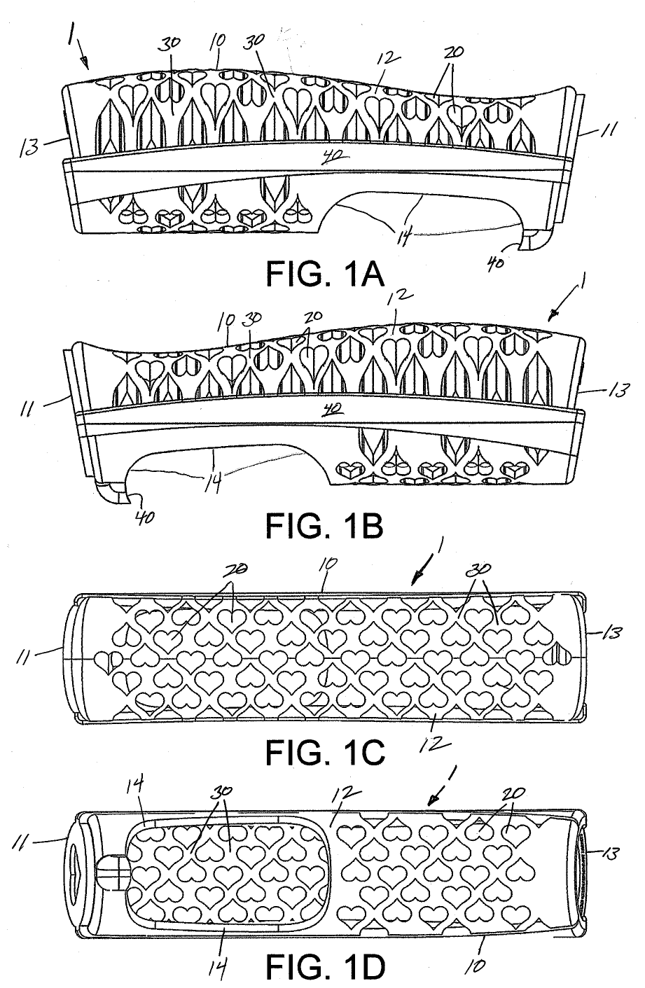

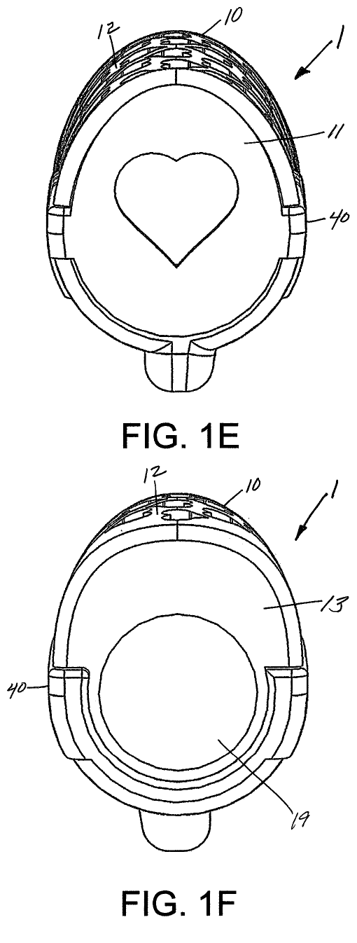

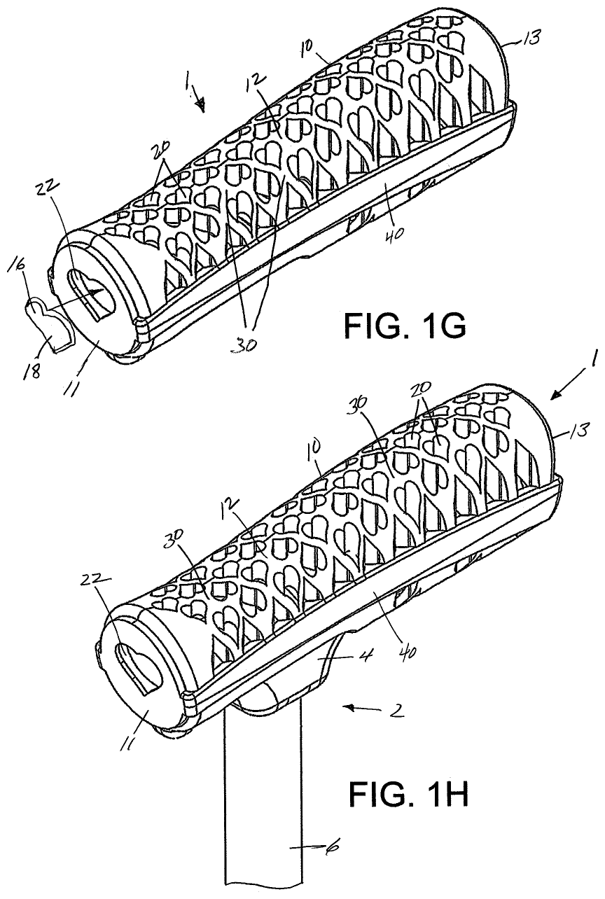

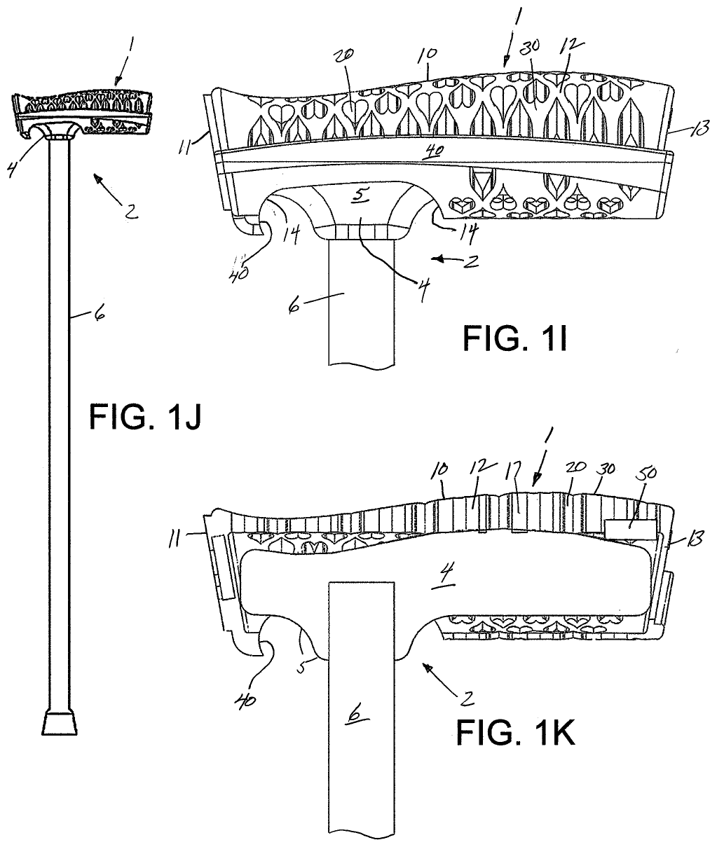

FIGS. 1A-1K depict a grip assist device fittable to a walking cane, and in FIGS. 1H-1K, the device 1 fitted to the walking cane 2. The walking cane 2 is comprised of a handle 4 and a support shaft 6 joined to and extending transversely from the handle 4. The grip assist device 1 is comprised of an elongated hollow body 10 formed of an elastomeric material. Suitable elastomeric materials include rubber polymers. Such a suitable rubber polymer deforms elastically when a stress is applied thereto, and reversibly deforms to its original shape and dimensions when the stress is removed. In one preferred embodiment, silicone rubber may be used to form the elongated hollow body 10, such as by injection molding, vacuum molding, or other suitable fabrication processes. Other elastomers having comparable coefficients of elasticity and durometers are suitable, including but not limited to thermoplastic elastomers and thermoplastic urethanes. In certain embodiments, the elastomeric material may be substantially optically clear, such that the appearance of the cane handle 4 is visible to a user of the cane 2 and device 1. In other embodiments, the elastomeric material may have a colored dye or filler to provide the device 1 in different colors.

The elongated hollow body 10 is comprised of an elastic mesh side wall 12 including perforations 20 interspersed with web sections 30, and an opening 14 in the sidewall 12 elastically deformable to receive the handle 4 of the cane 2 within the elongated hollow body 10. The perforations 20 may have various geometric shapes, including but not limited to circular, square, rectangular, polygonal, and elliptical shapes. In certain embodiments, the perforations do not extend completely through the sidewall 12 of the elongated body. Instead, the perforations are bounded at the inner surface of the sidewall by a thin film or membrane of the elastomeric material that is contiguous with the inner surface of the web sections 30. This configuration results in a continuous thin web of material at the inside surface of the sidewall 12. The configuration has been found to be advantageous in manufacturing repeatability and quality, and also strengthens to sidewall and prevents ripping of the sidewall 12 in fitting and using the device 1. The perforations 20 may have ornamental shapes such as the heart shapes shown in FIGS. 1A-1D and 1G. The perforations 20 may have central axes passing through a central axis of the elongated hollow body 10. Alternatively, as shown in the grip assist device 1 of FIGS. 1A-1D and 1G, the perforations 20 may be aligned with their central axes parallel to each other and in a vertical direction. This arrangement of perforations may result in easier fabrication and use of mold tooling in manufacturing of the grip assist device 1.

After the opening 14 is stretched over the cane handle 4, and the handle 4 is surrounded by the side wall 12 and end walls 11 and 13, the opening 14 reversibly deforms and surrounds the transversely extending support shaft 6 of the cane 2. If the handle 4 of the cane 2 includes a flared region 5, the opening 14 surrounds and conforms to the flared region 5 as well.

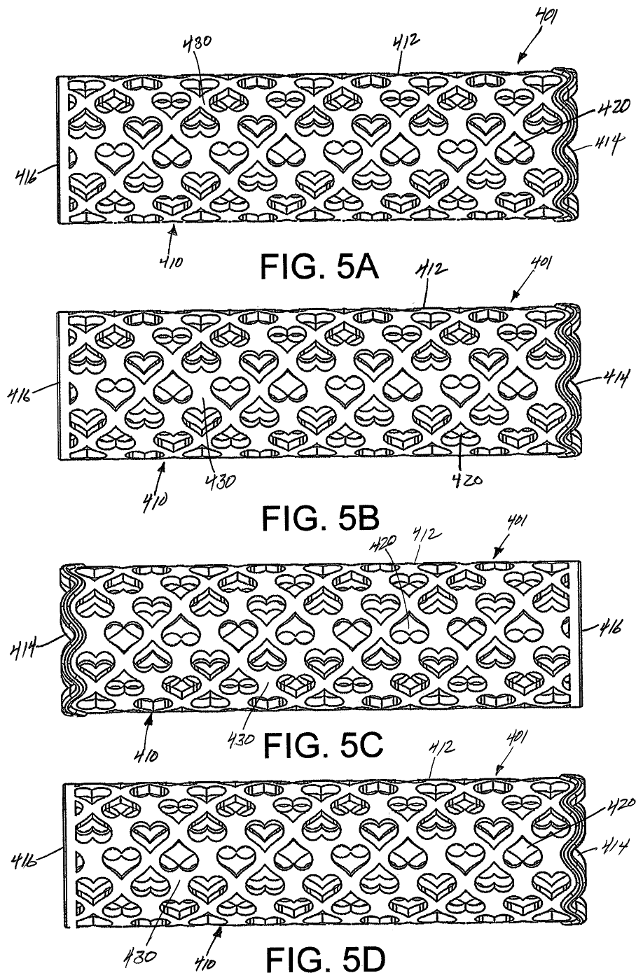

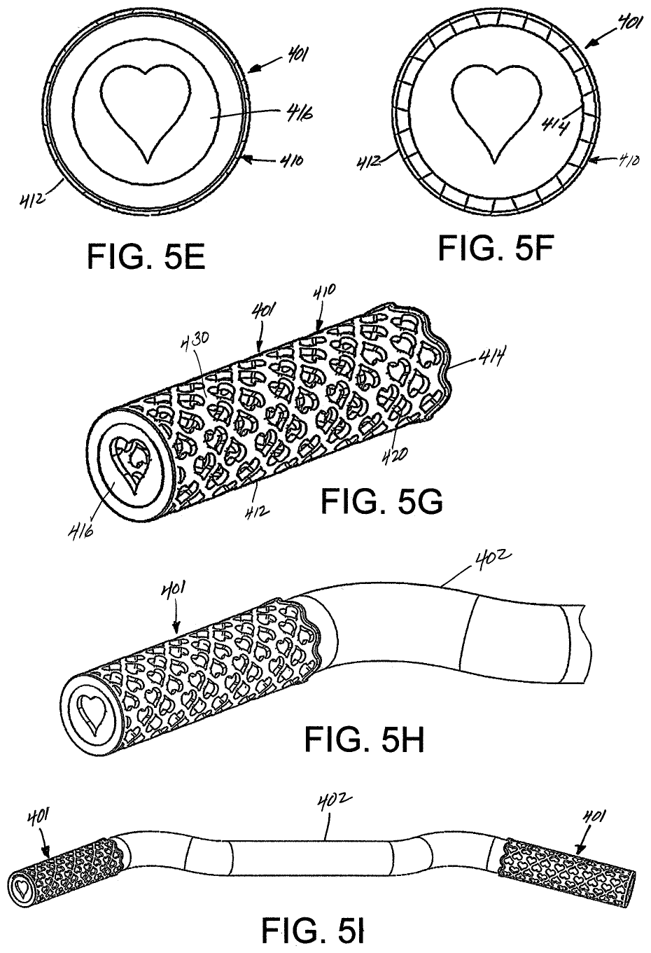

It is noted that the shape of the cane handle 4 may vary from that shown in FIG. 1K, with the gripping device still being capable of fitting to the cane handle 4, and functioning as intended. The upper region 17 of the side wall 12 may be provided with a greater thickness than the side and lower regions of the side wall 12. In that manner, greater cushioning of the handle 4 is provided by the grip assist device 1. This is advantageous because it is common for a user of the cane 2 to apply mostly a downward force on the upper surface of the cane handle 4 through the palm of the hand in order to obtain the needed support from the cane 2. In certain embodiments in which the cane handle is shorter than shown in FIG. 1K, the device 1 may include an opening 19 in the end 13 of the elongated body 10. In that manner, a pocket (not shown) is formed within the elongated body 10 for the storage of small objects, such as keys. At the location where the pocket is formed, the elongated body 10 may include other objects (not shown) integrated therein, such as electronics, lighting, a sensor, a camera, a label, user identification, user medical information, and/or tracking devices (e.g., an RFID chip). In one embodiment depicted in FIG. 5K, the pocket may contain an electronic communication and sensing device 50. The device 50 includes a sensor that detects when the device is gripped by a user. The device may include a processor that executes an algorithm including counting the number of times that the grip assist device 1 is gripped. This information can be stored in a memory of the electronic device 50 and also wirelessly transmitted to a network according to “Internet of Things” communication protocols. The electronic device 50 may also operate to provide an electrical stimulus to cause the release of additional antipathogenic additives on the surface 12 of the grip assist device 1. The electronic device 50 may also be used in data collection, including tracking of how many times the grip assist device 1 and cane 2 are used, and communicating an end of life and/or recommended replacement time.

It is further noted that in view of the shape and structure of the grip assist device 1 of FIGS. 1A-1G, the device may be fitted to other objects that have a shape similar to that of a cane handle. For example, the device 1 may be fitted to a lever-type door handle, such as a handle that is compliant with requirements of the Americans with Disabilities Act (ADA).

In certain embodiments, the grip assist device 1 may include a magnet 16 joined to the elongated hollow body 10. The magnet 16 may be joined to the end wall 11 of the elongated body 10, such as by an adhesive. Alternatively, as shown in FIG. 1G, the magnet 16 may be fitted into a pocket 22 in the end wall. Advantageously, the provision of a magnet 16 in the grip assist 1 enables the user to temporarily allow his or her cane 2 to be removably joined to a nearby metal surface, surface such as a car door or fender, while the user enters or exits a vehicle.

Alternatively or additionally, the grip assist device 1 may include a luminescent member 18 joined to the elongated hollow body. The luminescent material of the member 18 may be a plastic material, which includes a phosphor that can absorb light from a light source, and subsequently emit light that is visible in dark or dim environments. Advantageously, the provision of a luminescent member 18 in the grip assist 1 enables the user to first locate his or her cane 2 in a dark room, and make way to a light switch to turn on room lighting.

The magnet 16 and/or luminescent member 18 may have a simple shape such as that of a cylindrical disc (not shown), or an ornamental shape such as a heart shape as shown in FIG. 1G.

In certain embodiments, the elastomeric material of the elongated hollow body 10 may be made of a material having a high coefficient of friction. One suitable high friction is silicone rubber. It is difficult to slide an object made of silicone rubber on almost all non-lubricated solid surfaces. Other suitable high friction elastomers include polyurethane rubbers.

In such embodiments, the elongated hollow body 10 may be further comprised of a lateral protrusion 40 extending outwardly from a side region of the elongated hollow body 10. In that manner, with the elastomeric material of the elongated hollow body 10 having a high coefficient of friction, and a lateral protrusion 40, the cane 2 may be temporarily leaned against a wall (not shown) or other object (not shown). Due to the high coefficient of friction material, the cane 2 is less likely to slide and fall over, even if the user does not lean it against the wall in a relatively straight up position. Thus, almost any immobile solid object nearby to a user can function as a temporary storage location for the cane 2.

In certain embodiments, the elongated body 10 may further comprise a hook feature 24 to facilitate hanging of the cane 2 in a storage position or location, such as a clothes hanging hook on the back of a door, when the cane 2 is not in use.

The elastomeric material of the device 1 may include an antipathogenic material. The antipathogenic material may be provided in a coating adhered to an external surface of the elastic mesh side wall 12 of the elongated hollow body 10. alternatively or additionally, the antipathogenic material may be impregnated in the elastic mesh side wall 12 of the elongated hollow body 10.

In certain embodiments, the antipathogenic material may include silver, and/or antipathogenic forms of copper, such as copper sulfate and/or copper oxychloride. Alternatively or additionally, the antipathogenic material may include a zinc salt, such as zinc sulfate heptahydrate, anhydrous zinc sulfate, zinc acetate, and/or zinc nitrate; antimicrobial thermoplastic elastomers, which are effective against MRSA, E. Coli, Pseudomonas aeroginosa and Salmonella and many other harmful bacteria. The presence of atmospheric moisture facilitates the gradual release of silver ions and/or other antipathogenic additives from the elastomeric material. Other effective stimuli include exposure to sunlight or ultraviolet light, flexing and/or compression of the elastomeric material, and electrical charging and discharging of the elastomeric material. These ions and/or other additives interact with the micro-organisms to prevent a wide spectrum of bacteria from reproducing. Silver and other additives do not deteriorate and this controlled release of silver ions may last for the lifetime of the grip assist device 1. In certain embodiments, the antipathogenic material is formulated into the material concentration is consistent throughout the material and not solely on the surface, and the antipathogenic action of the grip assist device is prolonged as compared to antipathogenic material provided only on or near the surface of the side wall 12 of the device 1. Other antipathogenic materials are contemplated. The above antipathogenic materials are to be considered exemplary and not limiting. Antipathogenic materials that are effective against viruses, such as common cold, flu, and Covid viruses are particularly useful in the gripping device 1.

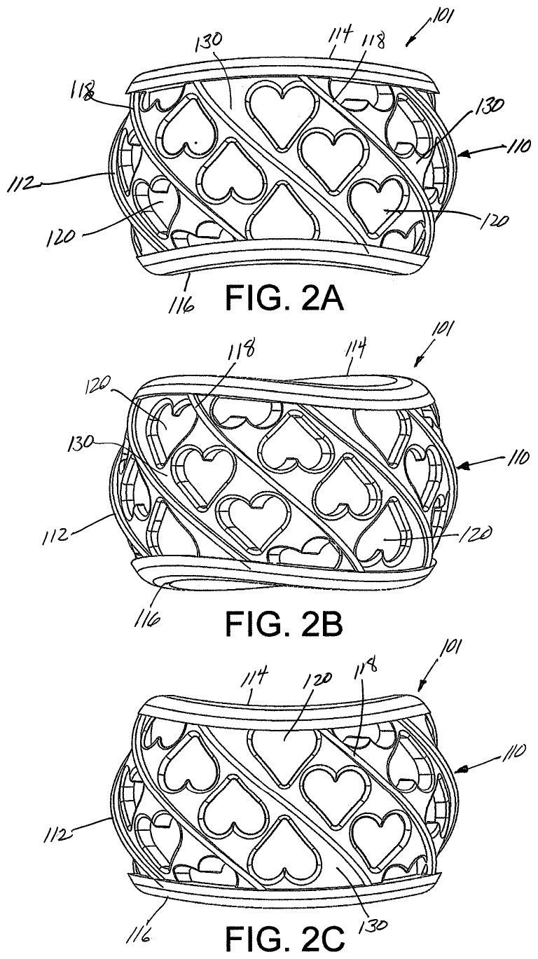

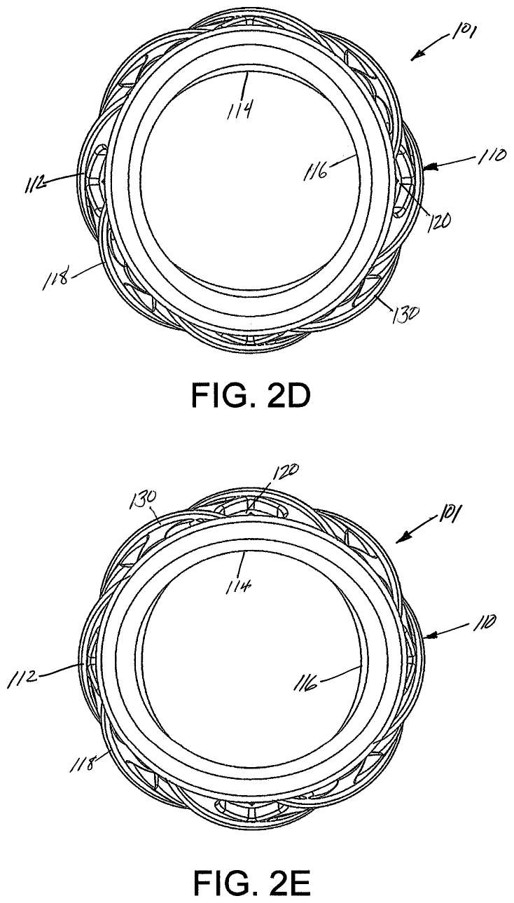

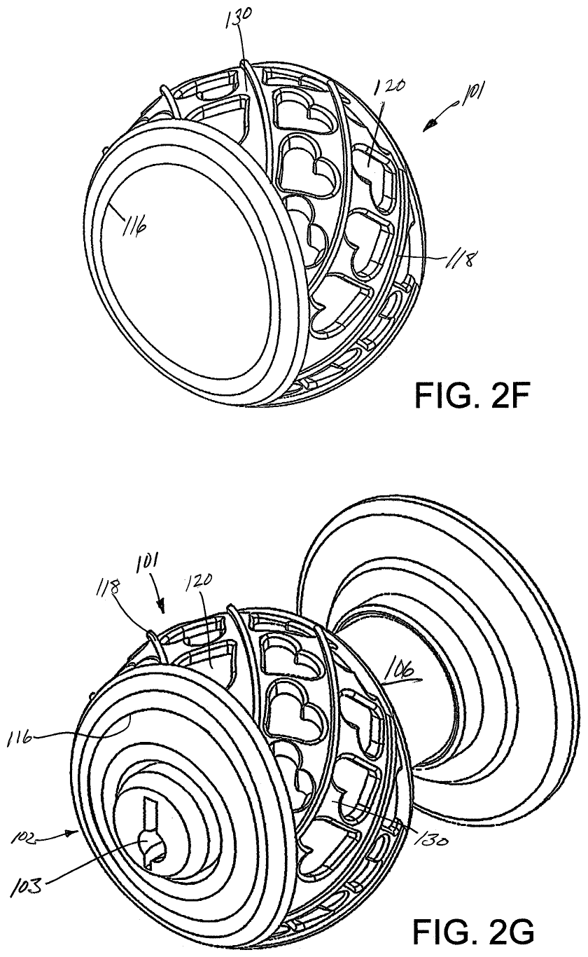

FIGS. 2A-2F depict a grip assist device fittable to a door knob, and in FIG. 2G, the device 101 fitted to the door knob 102. The grip assist device 101 is comprised of a hollow body 110 having a rearward opening 114 for receiving the door knob 102. The hollow body 110 is formed of an elastomeric material as described previously. The hollow body 110 is comprised of an elastic mesh side wall 112 including perforations 120 interspersed with web sections 130. The elastic mesh side wall 112 may further include ridges 118 on the exterior surface thereof which enhance the strength and comfort of the grip by a user.

After the opening 114 is stretched over the door knob 102, and the knob 102 is surrounded by the side wall 112, the opening 114 reversibly deforms and surrounds the transversely extending shaft 106 of the knob 102. The region of the side wall 112 that is proximate to the rearward opening 114 may form a round flange having a tapered cross-sectional geometry that enables further stretching than a flange of constant thickness. [CORRECT?]

For use on a door knob that includes a locking feature such as a keyhole 103, a rotatable tab (not shown) or a push button (not shown), the device 101 may include a forward opening 116 that provides access to the locking feature. The hollow body 110 of the device 101 may include an antipathogenic material as described previously. The perforations 120 may have various geometric shapes, including but not limited to circular, square, rectangular, polygonal, and elliptical shapes. The perforations 120 may have ornamental shapes such as the heart shapes shown in FIGS. 2A-2G.

The perforations 120 in combination with the elastic properties of the elastomeric material enable the side wall 112 of the device 101 to conform to various door knob shapes and sizes. In embodiments in which the elastomeric material is optically clear, the underlying style and structure of the door knob 102 are not obscured.

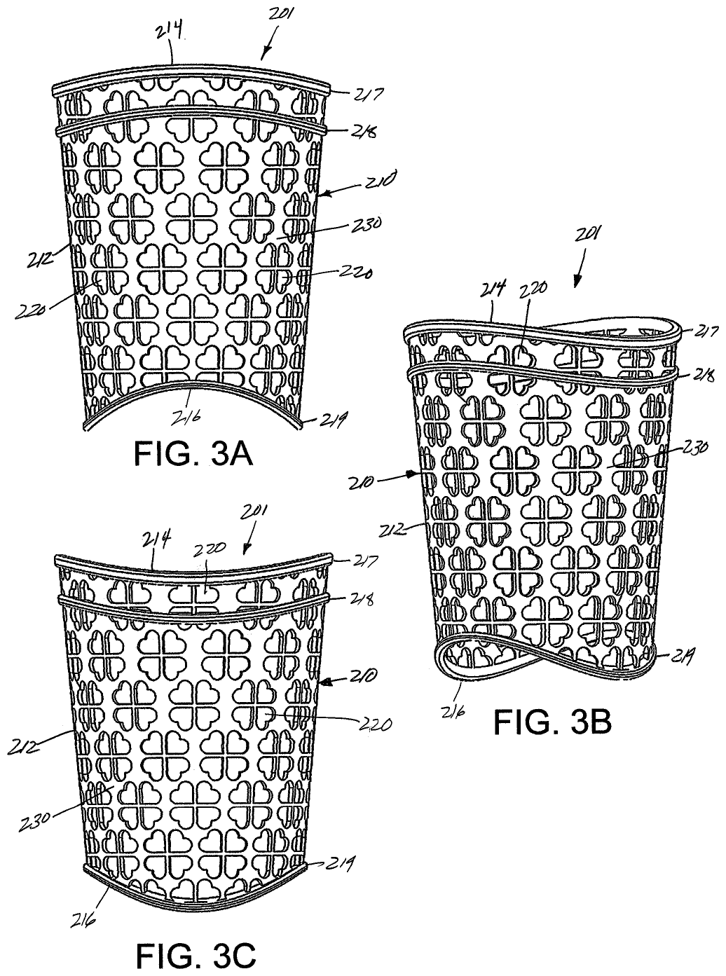

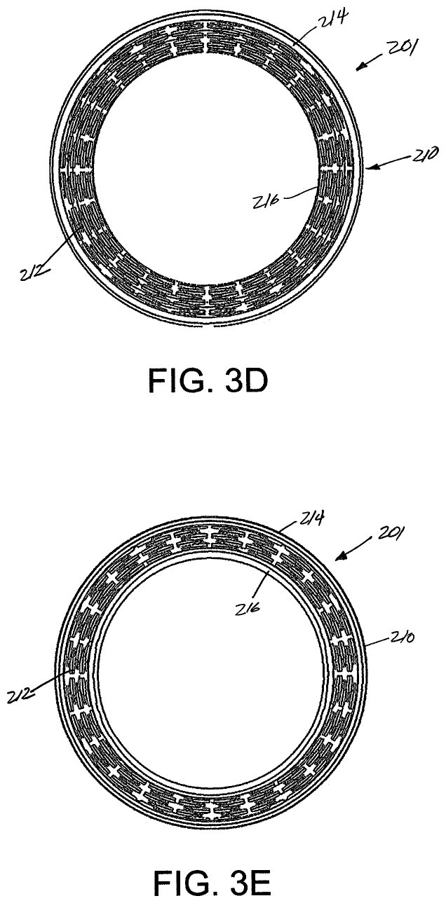

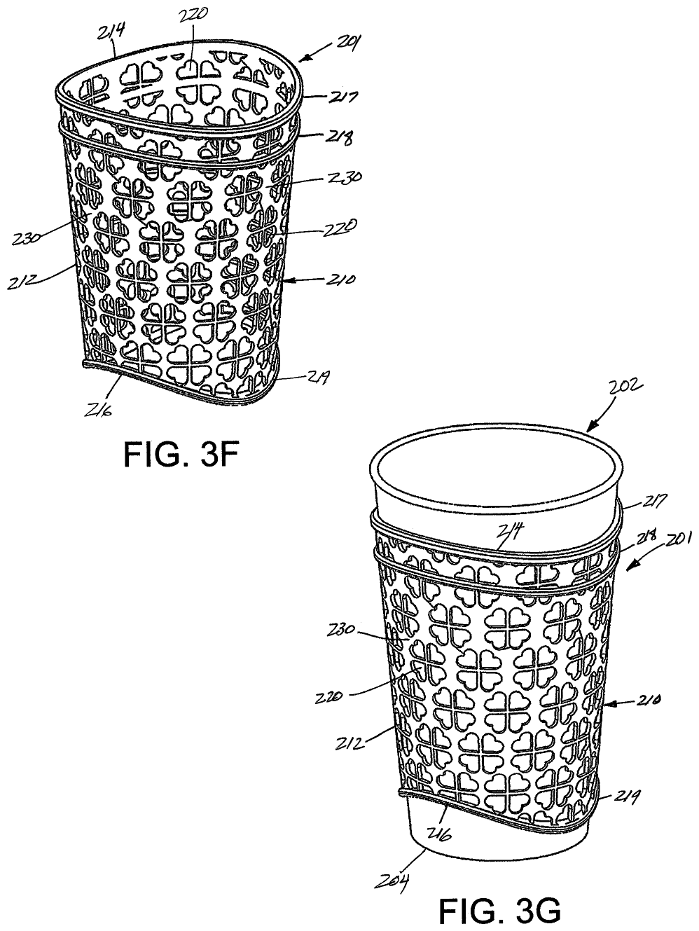

FIGS. 3A-3F depict a grip assist device fittable to a beverage cup, and in FIG. 3G, the device 201 fitted to the beverage cup 202. The grip assist device 201 is comprised of a hollow body 210 having a top opening 214 for receiving the bottom end 204 of the cup 202. The hollow body 210 is formed of an elastomeric material as described previously. The hollow body 210 is comprised of an elastic mesh side wall 212 including perforations 220 interspersed with web sections 230. The elastic mesh side wall 212 may further include ridges 217, 218, and 219 on the exterior surface thereof which enhance the strength and comfort of the grip by a user. The middle ridge 218 may serve as a thumb rest feature when gripping the device 202 and a cup 202. After the top opening 214 is passed over the bottom end 204 of the cup 202, the cup 202 is further forced through the hollow body 210 until the bottom end 204 of the cup 202 extends through the bottom opening 216, and the cup 202 is surrounded by the side wall 112 of the grip assist device 201. The middle region of the side wall 212 may be of greater thickness than the upper and lower end regions to enhance grip by a user and insulation from hot or cold liquid in the cup 202.

The hollow body 210 of the device 201 may include an antipathogenic material as described previously. The perforations 220 may have various geometric shapes, including but not limited to circular, square, rectangular, polygonal, and elliptical shapes. The perforations 220 may have ornamental shapes such as the heart shapes shown in FIGS. 3A-3F, which may be arrayed as shown to form shamrock images.

The perforations 220 in combination with the elastic properties of the elastomeric material enable the side wall 212 of the device 201 to conform to various cup shapes, tapers, and sizes. In embodiments in which the elastomeric material is optically clear, the underlying style and structure of the cup 202 are not obscured.

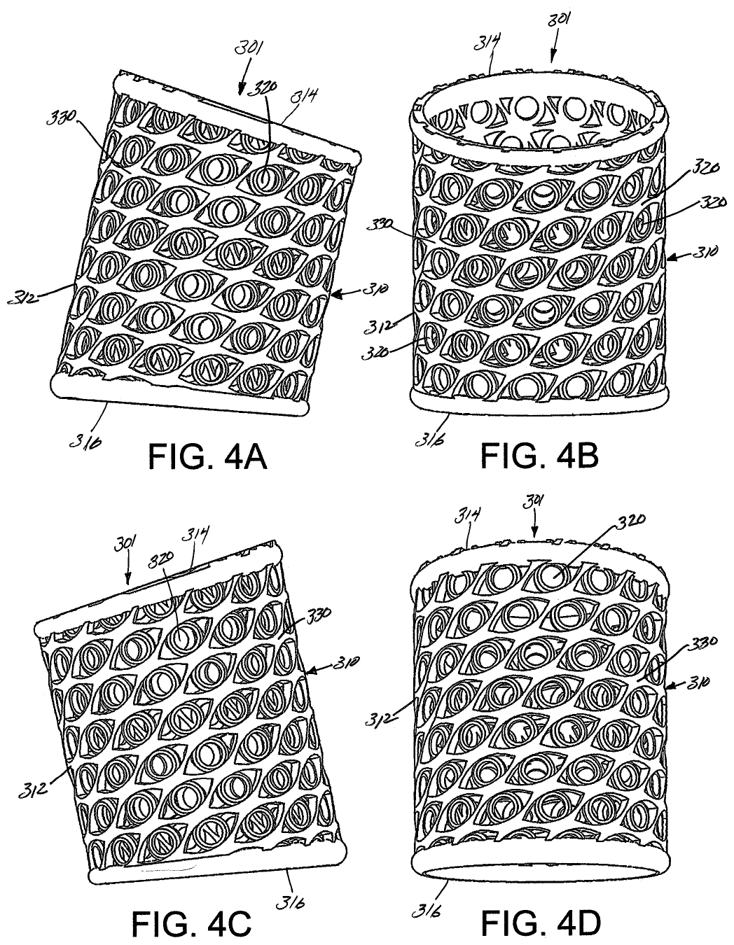

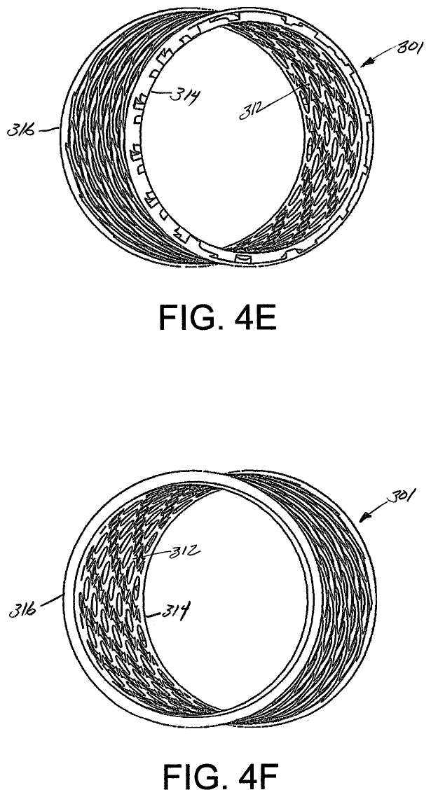

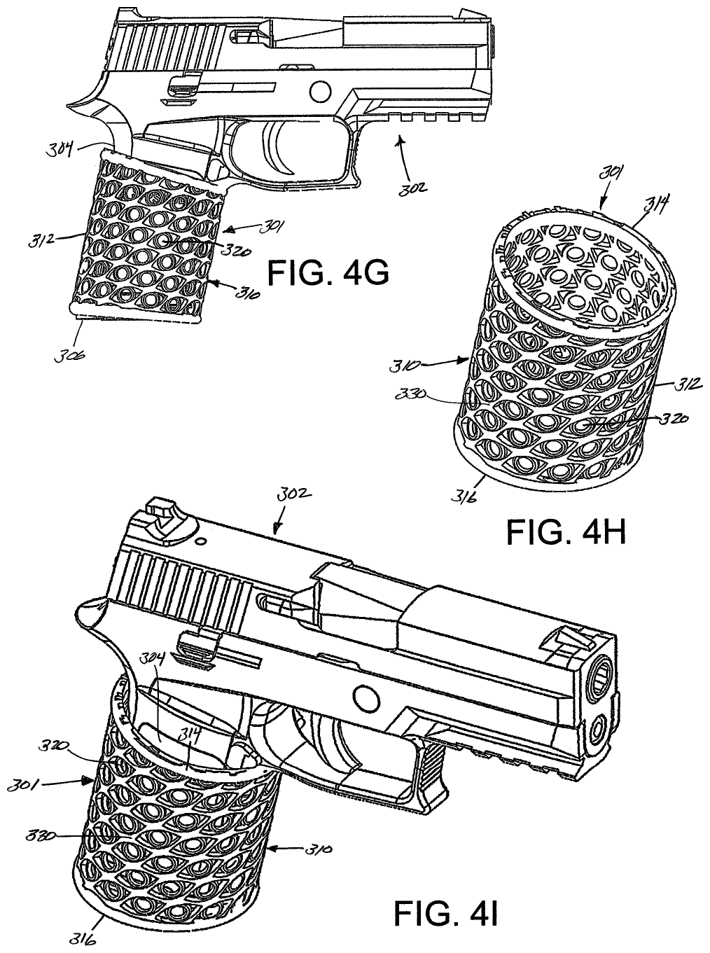

FIGS. 4A-4F and 4H depict a grip assist device fittable to a handgun, and in FIGS. 4G, 4I, and 4J, the device 301 fitted to the handgun 302. The grip assist device 301 is comprised of a hollow body 310 having a top opening 314 for receiving the butt 304 of a handgun 302. The hollow body 310 is formed of an elastomeric material as described previously. The hollow body 310 is comprised of an elastic mesh side wall 312 including perforations 320 interspersed with web sections 330.

After the top opening 314 is stretched over the bottom 306 of the butt 304, the butt 304 is further forced through the hollow body 310 until the butt 304 of the gun 302 extends through the bottom opening 316, and the butt 304 is surrounded by the side wall 312 of the grip assist device 301.

The hollow body 310 of the device 101 may include an antipathogenic material as described previously. The perforations 320 may have various geometric shapes, including but not limited to circular, square, rectangular, polygonal, and elliptical shapes. The perforations 320 may have ornamental shapes such as the eye-like shapes shown in FIGS. 4A-4F.

The perforations 320 in combination with the elastic properties of the elastomeric material enable the side wall 312 of the device 301 to conform to various gun butt shapes and sizes, to improve the purchase of the handgun 402 when drawing it from a holster, and to reduce the impact of recoil on a user's hand when firing the handgun 402.

FIGS. 5A-5G depict a grip assist device fittable to a bicycle handlebar, and in FIGS. 5H, and 5I, the device 401 fitted to the handlebar 402. The grip assist device 401 is comprised of a hollow body 410 having an end opening 414 for receiving the outer end of a handlebar 402. The hollow body 410 is formed of an elastomeric material as described previously. The hollow body 410 is comprised of an elastic mesh side wall 412 including perforations 420 interspersed with web sections 430. After the end opening 414 is passed over the outer end of the handlebar 402, the handlebar 402 is further forced through the hollow body 410 until the outer end of the handlebar 402 contacts the closed end 416 of the hollow body 410 and the handlebar 402 is surrounded by the side wall 412 of the grip assist device 401.

The hollow body 410 of the device 401 may include an antipathogenic material as described previously. The perforations 420 may have various geometric shapes, including but not limited to circular, square, rectangular, polygonal, and elliptical shapes. The perforations 420 may have ornamental shapes such as the heart shapes shown in FIGS. 5A-5D and 5G. The perforations 420 in combination with the elastic properties of the elastomeric material enable the side wall 412 of the device 401 to conform to hand held objects including but not limited to knives, golf clubs, sports racquets, sports paddles, and bats.

The grip assist devices of the present disclosure are advantageous because they are reusable, and may be removed, washed, disinfected, autoclaved, and reinstalled on the respective devices that they are designed for. Additionally, they can be applied over an existing object without damage to the original finish or change in condition of the object.

It is therefore apparent that there has been provided, in accordance with the present disclosure, gripping devices for retrofitting onto hand-held and hand-operated articles and apparatus. The foregoing description of technology and the invention is merely exemplary in nature of the subject matter, manufacture, and use of the invention and is not intended to limit the scope, application, or uses of any specific invention claimed in this application or in such other applications as may be filed claiming priority to this application, or patents issuing therefrom. The following definitions and non-limiting guidelines must be considered in reviewing the description.

The headings in this disclosure (such as “Background” and “Summary”) and sub-headings used herein are intended only for general organization of topics within the present technology, and are not intended to limit the disclosure of the present technology or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

To the extent that other references may contain similar information in the Background herein, said statements do not constitute an admission that those references are prior art or have any relevance to the patentability of the technology disclosed herein. Any discussion in the Background is intended merely to provide a general summary of assertions.

The description and specific examples, while indicating embodiments of the technology disclosed herein, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

Unless otherwise specified, relational terms used in the present disclosure should be construed to include certain tolerances that those skilled in the art would recognize as providing equivalent functionality. By way of example, the term perpendicular is not necessarily limited to 90.00°, but also to any variation thereof that those skilled in the art would recognize as providing equivalent functionality for the purposes described for the relevant member or element. Terms such as “about” and “substantially” in the context of configuration relate generally to disposition, location, and/or configuration that is either exact or sufficiently close to the location, disposition, or configuration of the relevant element to preserve operability of the element within the invention while not materially modifying the invention. Similarly, unless specifically specified or clear from its context, numerical values should be construed to include certain tolerances that those skilled in the art would recognize as having negligible importance, as such do not materially change the operability of the invention.

As referred to herein, all compositional percentages are by weight of the total composition, unless otherwise specified. As used herein, the words “comprise,” “include,” contain,” and variants thereof are intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

In the following description, numerous details are set forth to provide an understanding of the disclosed apparatus and methods. However, it will be understood by those skilled in the art that the apparatus and methods covered by the claims may be practiced without these details and that numerous variations or modifications from the specifically described embodiments may be possible and are deemed within the claims. For example, wherever the term “comprising” is used, embodiments and/or components where “consisting essentially of” and “consisting of” are also explicitly disclosed herein and are part of this disclosure. An example of “consisting essentially of” may be with respect to the composition of a conduit: a conduit consisting essentially of carbon steel means there may be a minor portions or trace amounts of metals, oxides, and other chemical species that are noble metals, such chromium, platinum, and the like, and a conduit consisting essentially of noble metal may have trace amounts of iron, iron oxides, carbon, and other metal oxides. An example of “consisting of” may be a burner made up of components that are one or more carbon steels and no noble metals or ceramic materials, or conduits made up of only noble metals. Another example of “consisting essentially of” may be with respect to particulate feedstock that consists essentially of inorganic feedstock, meaning that a minor portion, perhaps up to 10, or up to 5, or up to 4, or up to 3, or up to 2, or up to 1 wt. percent may be organic. An example of methods and systems using the transition phrase “consisting of” includes those where only burners having liquid-cooled jackets are used, with no gas-cooled jackets, or vice versa. The term “comprising” and derivatives thereof is not intended to exclude the presence of any additional component, step or procedure, whether or not the same is disclosed herein. In order to avoid any doubt, all compositions, apparatus, systems, and methods claimed herein through use of the term “comprising” may include any additional component, step, or procedure unless stated to the contrary. In contrast, the term, “consisting essentially of” excludes from the scope of any succeeding recitation any other component, step or procedure, excepting those that are not essential to operability. The term “consisting of” excludes any component, step or procedure not specifically delineated or listed. The term “or”, unless stated otherwise, refers to the listed members individually as well as in any combination

All numbers disclosed herein are approximate values, regardless whether the word “about” or “approximate” is used in connection therewith. They may vary by 1%, 2%, 5%, and sometimes, 10 to 20%.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting ingredients, components or process steps, the Applicants specifically envision embodiments consisting of, or consisting essentially of, such ingredients, components or processes excluding additional ingredients, components or processes (for consisting of) and excluding additional ingredients, components or processes affecting the novel properties of the embodiment (for consisting essentially of), even though such additional ingredients, components or processes are not explicitly recited in this application. For example, recitation of a composition or process reciting elements A, B, and C specifically envisions embodiments consisting of, and consisting essentially of, A, B, and C, excluding an element D that may be recited in the art, even though element D is not explicitly described as being excluded herein.

Having thus described the basic concept of the invention, it will be apparent to those skilled in the art that the foregoing detailed disclosure is intended to be presented by way of example only, and is not limiting. Various alterations, improvements, and modifications will occur to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be expressly stated in the claims.

We claim:

1. A grip assist device fittable to a walking cane comprising a handle and a support shaft joined to and extending transversely from the handle, the grip assist device comprising an elongated hollow body formed of an elastomeric material and comprised of an elastic mesh side wall including perforations interspersed with web sections, and an opening in the sidewall elastically deformable to receive the handle of the cane within the elongated hollow body and to reversibly deform and surround the transversely extending support shaft of the cane.

2. The grip assist device of claim 1, wherein the elastomeric material includes an antipathogenic material.

3. The grip assist device of claim 2, wherein the antipathogenic material is provided in a coating adhered to an external surface of the elastic mesh side wall of the elongated hollow body.

4. The grip assist device of claim 2, wherein the antipathogenic material is impregnated in the elastic mesh side wall of the elongated hollow body.

5. The grip assist device of claim 2, wherein the antipathogenic material includes copper, copper alloys and/or silver.

6. The grip assist device of claim 1, further comprising a magnet joined to the elongated hollow body.

7. The grip assist device of claim 1, further comprising a luminescent member joined to the elongated hollow body.

8. The grip assist device of claim 1, wherein the elastomeric material of the elongated hollow body is made of a material having a high coefficient of friction, and the elongated hollow body is further comprised of a lateral protrusion extending outwardly from a side region of the elongated hollow body.

9. The grip assist device of claim 1, wherein the elongated body further comprises a hook feature.

10. A grip assist device fittable to a hand-held object, the grip assist device comprising a hollow body having at least one opening for receiving the object, the hollow body formed of an elastomeric material, including an antipathogenic material. and comprised of an elastic mesh side wall including perforations interspersed with web sections.

11. The grip assist device of claim 10, wherein the antipathogenic material is provided in a coating adhered to an external surface of the elastic mesh side wall of the elongated hollow body.

12. The grip assist device of claim 10, wherein the antipathogenic material is impregnated in the elastic mesh side wall of the elongated hollow body.

13. The grip assist device of claim 10, wherein the antipathogenic material includes copper.
